(19) United States
(12) Patent Application Publication
(10) Pub. No.: US 2024/0245334 A1
(43) Pub. Date: Jul. 25, 2024

(54) SYSTEMS AND METHODS FOR DIAGNOSING ATTENTION DISORDER, EMOTIONAL DYSREGULATION, AND IMPULSIVE DYSREGULATION

(71) Applicant: Mindtension LTD, Nir-Am (IL)

(72) Inventors: Avraham AVITAL, Sarid (IL); Zev BRAND, Modiin (IL)

(73) Assignee: Mindtension LTD, Nir-Am (IL)

(21) Appl. No.: 18/290,416

(22) PCT Filed: May 19, 2022
(86) PCT No.: PCT/IL2022/050523

§ 371 Date: Nov. 13, 2023

(30) Foreign Application Priority Data
May 19, 2021 (IL) 283304

(51) Int. Cl. A61B 5/16; A61B 5/00; A61B 5/389; A61B 5/395

(52) U.S. Cl. CPC A61B 5/168; A61B 5/162; A61B 5/389; A61B 5/395; A61B 5/6821

(57) ABSTRACT

A method for diagnosing attention disorder is disclosed. The method comprising: providing to a user a predetermined continuous background noise; providing to the user, a first audio stimulation comprising a single sound in a first known intensity; detecting a first startle response; providing to the user, a second audio stimulation comprising two sounds, a first sound in a second intensity and a second sound having the first intensity, wherein the second intensity is selected to be higher than the background noise in an amount that allows the first sound to be noticed by the user; detecting a second startle response; diagnosing an attention disorder is based on the inhibition between the second startle response and the first startle response and sending a signal comprising the diagnosis to an external computing device.

### CROSS REFERENCE TO RELATED APPLICATIONS

This application claims the benefit of priority of IL Patent Application No. 283304, filed on May 19, 2021, the contents of which are all incorporated herein by reference in their entirety.

### FIELD OF THE INVENTION

The invention generally relates to systems and methods for diagnosing attention disorder, emotional dysregulation, and impulsive dysregulation. More specifically the invention relates to systems and methods for diagnosing attention disorder, emotional dysregulation, and impulsive dysregulation by detecting startle responses.

### BACKGROUND OF THE INVENTION

Diagnosing attention disorders, such as, attention deficit hyperactivity disorder (ADHD) is conducted by professionals such as, a child or adult psychiatrist, a neurologist, a pediatrician specialist in children's health, a learning disability specialist, and a social worker or an occupational therapist with expertise in ADHD.

There's no simple objective test to determine whether an adult or a child has ADHD, but the specialist can make the diagnosis after a detailed assessment. The heteroanamnesis assessment may include a physical examination, which can help rule out other possible causes for the symptoms, a series of interviews with the examinee, and interviews or reports from other significant people, such as partners, parents, and teachers. These methods rely heavily on the professionalism of the specialist and the cooperation of the examinee and the other significant people.

Similarly, diagnosing emotional and impulsive dysregulation also requires the assessment of a specialist. The accuracy of the assessment, again, relies of the professionalism of the specialist and the cooperation of the examinee.

In 1975 an attempt was made to diagnose schizophrenia using pre-pulse inhibition (PPI). PPI is a neurological phenomenon in which a weaker pre-stimulus (pre-pulse) inhibits the reaction of an organism to a subsequent strong reflex-eliciting stimulus (pulse), often using the startle reflex. The stimuli are usually acoustic. The reduction or lack of reduction of the amplitude of the startle reflects the ability of the nervous system to temporarily adapt to a strong sensory stimulus when a preceding weaker signal is given to warn the organism (e.g., ears). In the 1975 test, known in the art as, the “San Diago” test, examinees were given acoustical stimulus and pre-stimulus and the startle response was measured using Electromyography (EMG). The test results were not conclusive and the method was abandoned.

Accordingly, there is a need for objective, reliable methods for diagnosing attention disorders, emotional dysregulation, and impulsive dysregulation, that are independent of the professionalism of the specialist and/or the cooperation of the examinee. In some embodiments, such methods may benefit from detecting PPI.

### SUMMARY OF THE INVENTION

Some aspects of the invention may be directed to a method for diagnosing attention disorder, comprising: providing to a user a predetermined continuous background noise; providing to the user, a first audio stimulation comprising a single sound in a first known intensity; detecting a first startle response; providing to the user, a second audio stimulation comprising two sounds, a first sound in a second intensity and a second sound having the first intensity, wherein the second intensity is selected to be higher than the background noise in an amount that allows the first sound to be noticed by the user; detecting a second startle response; diagnosing an attention disorder is based on the inhibition between the second startle response and the first startle response and sending a signal comprising the diagnosis to an external computing device.

In some embodiments, the single sound of the first audio stimulation and the second sound of the second audio stimulation are provided in a known first duration. In some embodiments, the first sound of the second audio stimulation is provided in a second known duration.

In some embodiments, the first startle response and the second startle response are detected using electromyography (EMG) electrodes placed on an Orbicularis Oculi muscle of the user. In some embodiments, detecting each of the first startle response and detecting the second startle response comprises detecting at least one of the maximum intensity of an EMG signal, the rise in time of the EMG signal intensity, and the decay time of the EMG signal intensity and any other EMG related feature such as but not limited to-latency, maximum response decay time, rise time, response pattern and more.

In some embodiments, the first startle response and the second startle response are detected using a stream of images of at least a portion of the user's face. In some embodiments, detecting each of the first startle response and detecting the second startle response comprises detecting a maximum movement of an eyelid of at least one eye detected from the stream of images.

In some embodiments, the first known intensity is between 4-400 dB. In some embodiments, the predetermined continuous background noise is between 40-80 dB. In some embodiments, the second sound is provided 30-600 milliseconds after the first sound. In some embodiments, the first and second audio stimulations are provided to both ears of the user situationally using earphones. In some embodiments, the duration of the first audio stimulation and the second sound in the second audio stimulation is 10-500 milliseconds. In some embodiments, the duration of the second sound is 2-400 milliseconds.

Some additional aspects of the invention may be directed to a method for diagnosing emotional dysregulation and/or impulsive dysregulation, comprising: providing to a user, a first audio stimulation comprising a first sound in a first known intensity; detecting a first startle response; providing to the user, a second audio stimulation comprising a second sound in a second known intensity higher than the first intensity; detecting a second startle response; diagnosing emotional dysregulation and/or impulsive dysregulation if the second startle response is equal to or lower than the first startle response; and sending a signal comprising the diagnosis to an external computing device. In some aspects of the invention, diagnosing emotional dysregulation and/or impulsive dysregulation is comprised from a series of auditory stimulations in various intensities that generates startle responses. These responses are then classified using machine learning to identify specific patterns unique to emotional dysregulation and/or impulsive dysregulation compared to healthy participants. More specifically, machine learning parameters include the following dimensions but limited to Linear coefficients between startle responses for each intensity, non-linear coefficients between startle responses for each intensity, eye-blink frequency patterns, maximal peak response for each intensity, average peak response for each intensity, minimal peak response for each intensity, the latency of the peak response for each intensity.

In some embodiments, the method may further include providing a user with predetermined continuous background noise. In some embodiments, the first sound of the first audio stimulation and the second sound of the second audio stimulation are provided in a known first duration. In some embodiments, the first sound of the second audio stimulation is provided in a second known duration.

In some embodiments, the first startle response and the second startle response are detected using electromyography (EMG) electrodes placed on an Orbicularis Oculi muscle of the user. In some embodiments, detecting each of the first startle responses and detecting the second startle response comprises detecting at least one of: the maximum intensity of an EMG signal, the rise in time of the EMG signal intensity, and the decay time of the EMG signal intensity, and any other EMG related feature such as but not limited to-latency, maximum response, rise time, response pattern and more.

In some embodiments, the first startle response and the second startle response are detected using a stream of images of at least a portion of the user's face. In some embodiments, detecting each of the first startle responses and detecting the second startle response comprises detecting a maximum movement of an eyelid of at least one eye detected from the stream of images.

In some embodiments, the first known intensity is between 4-400 dB and the second known intensity is between 4-400 dB. In some embodiments, the first and second stimulations are provided to both ears of the user situationally using earphones. In some embodiments, the duration of the first audio stimulation and the second audio stimulation is 10-500 milliseconds. In some embodiments, the inter-stimulus interval is 30-600 milliseconds.

In some embodiments, the method may further include providing to the user, a third audio stimulation comprising a third sound in a third known intensity higher than the second intensity and detecting a third startle response. In some embodiments, diagnosing the emotional dysregulation and/or impulsive dysregulation includes determining if the third startle response is equal to or lower than the second startle response.

Some additional aspects of the invention may be directed to a method for diagnosing schizophrenia, comprising: providing to a user a predetermined continuous background noise; providing to the user, a first audio stimulation comprising a single sound in a first known intensity; detecting a first startle response; providing to the user, a second audio stimulation comprising two sounds, a first sound in a second intensity and a second sound having the first intensity, wherein the second intensity is selected to be higher than the background noise in an amount that allows the first sound to be noticed by the user; detecting a second startle response; diagnosing schizophrenia if the ratio between the second startle response and the first startle response is higher than 10%; and sending a signal comprising the diagnosis to an external computing device.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A is a block diagram, depicting a system for diagnosing attention disorder and/or emotional dysregulation and/or impulsive dysregulation, according to some embodiments;

FIG. 1B is a block diagram, depicting a computing device which may be included in a system for diagnosing attention disorder and/or emotional dysregulation and/or impulsive dysregulation, according to some embodiments;

FIG. 2 is a block diagram of a non-limiting example for a system for diagnosing attention disorder and/or emotional dysregulation and/or impulsive dysregulation, according to some embodiments;

FIG. 3 is a flowchart of a method for diagnosing attention disorder according to some embodiments of the invention;

FIGS. 4A and 4B are graphs showing detected first and second startle responses of a first examinee, respectively, according to some embodiments of the invention;

FIGS. 5A and 5B are graphs showing detected first and second startle responses of a second examinee, respectively, according to some embodiments of the invention;

FIGS. 6A and 6B are tables showing scoring given to two examinees following a diagnosis of attention disorder according to some embodiments of the invention;

FIG. 7 is a flowchart of a method for diagnosing emotional dysregulation and/or impulsive dysregulation according to some embodiments of the invention;

FIGS. 8A and 8B are graphs showing startle responses at 3 different intensity levels for a normal examinee and an examinee having emotional dysregulation and/or impulsive dysregulation according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to a particular order or sequence. Additionally, some of the described method embodiments or elements thereof can occur or be performed simultaneously, at the same point in time, or concurrently.

Embodiments of the present invention disclose methods and a system for diagnosing attention disorder and/or emotional dysregulation and/or impulsive dysregulation. In some embodiments, a method for diagnosing attention disorder may detect the PPI neurological phenomenon in examinees following acoustical stimuli. In some embodiments, the method may provide an objective independent diagnosis to ADHD in both adults and children. Since such a method does not require the cooperation of the examinee, the method can be performed already on toddlers and babies. Therefore, treating ADHD can start at a very early age, when the brains is still flexible.

Furthermore, methods of diagnosing attention disorder according to embodiments of the invention may use to assess the performance of a person in life endangering professions, such as combat/civil pilots and physicians. The inattention of combat/civil pilots may lead to a high prevalence of life-endangering events (misjudgment, etc.). Inattention among physicians (overload, etc.) may lead to mal-treatment and medical errors.

In some embodiments, a method for diagnosing emotional dysregulation and/or impulsive dysregulation may use another neurological phenomenon following the provision of two or more (e.g., three) consecutive acoustic stimuli. Such a method can diagnose the response pattern to the various stimuli, indicating the presence of dysregulation (e.g., indifference to the stimuli intensity).

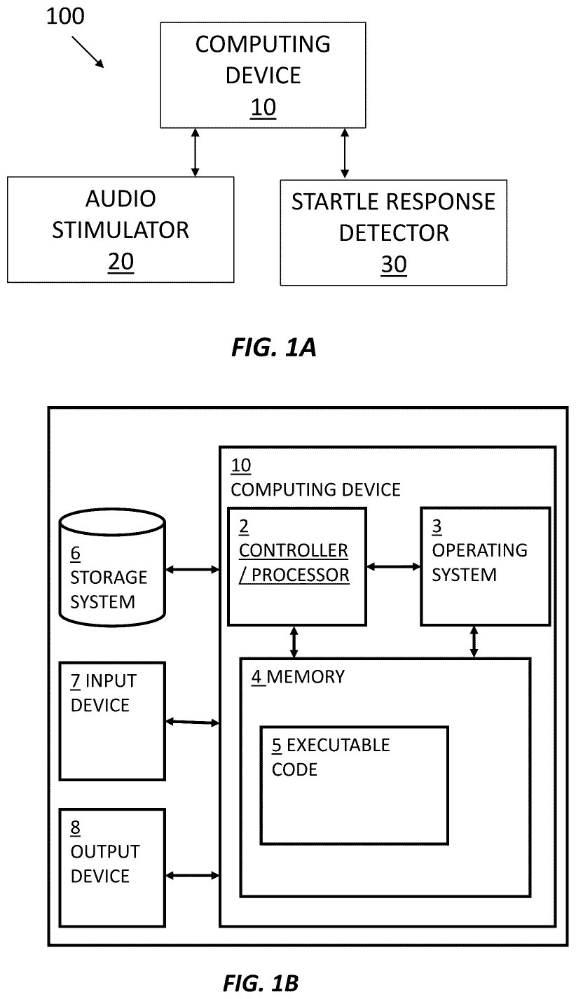

Reference is now made to FIG. 1A which is a block diagram of a system for diagnosing attention disorder, emotional dysregulation, and/or impulsive dysregulation according to some embodiments of the invention. A system 100 may include a computing device 10, for example, computing device 10, illustrated and discussed with respect to FIG. 1B. In some embodiments, system 100 may further include an audio stimulator 20 and a startle response detector 30.

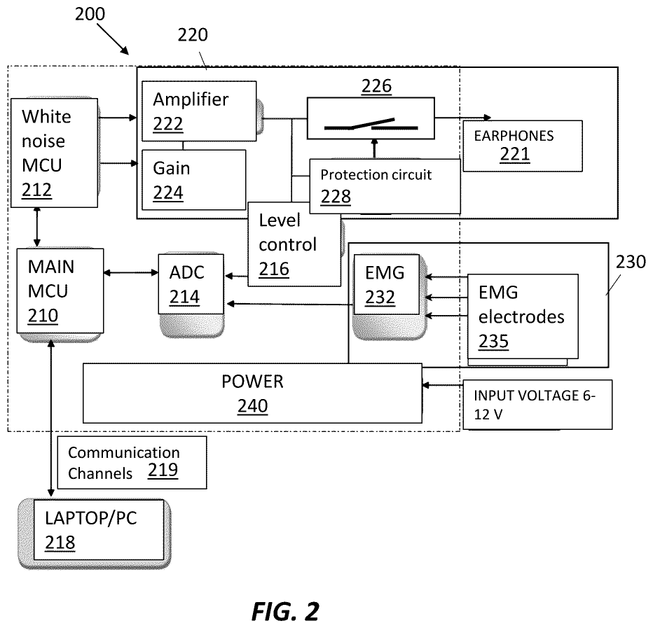

Audio stimulator 20 may include an audio signal generator and audio signal provider, for example, earphones, loudspeakers, and the like. In some embodiments, computing device 10 may control audio stimulator 20 to provide audio signals (e.g., to an examinee) having known intensities and/or known duration. A non-limiting example for an audio stimulator 220 is discussed with respect to the block diagram of FIG. 2.

Startle response detector 30 may include any device that may detect the startle response of the examinee. For example, startle response detector 30 may include electromyography (EMG) and EMG electrodes to be placed on an Orbicularis Oculi muscle of the examinee. In another example, startle response detector 30 may include a camera for capturing a stream of images of at least a portion of the examinee's face, for example, an eyelid movement of at least one eye.

Reference is now made to FIG. 1B, which is a block diagram depicting a computing device, which may be included within an embodiment of a system for diagnosing attention disorder, emotional dysregulation, and/or impulsive dysregulation, according to some embodiments.

Computing device 10 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 10 may be included in, and one or more computing devices 10 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random Access Memory (RAM), a read-only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under the control of operating system 3. For example, executable code 5 may be an application that may diagnose attention disorder, emotional dysregulation, and/or impulsive dysregulation as further described herein. Although, for the sake of clarity, a single item of executable code 5 is shown in FIG. 1B, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a microcontroller or chip as known in the art, a hard disk drive, a CD-Recordable (CD-R) drive, a Blu-ray disk (BD), a universal serial bus (USB) device or other suitable removable and/or fixed storage unit. Data for diagnosing attention disorder, emotional dysregulation and/or impulsive dysregulation may be stored in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 10 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 10 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

According to some embodiments of the invention, system 100 may be implemented as a software module, a hardware module or any combination thereof. For example, system 100 may be or may include a computing device such as element 10 of FIG. 1B, and may be adapted to execute one or more modules of executable code (e.g., element 5 of FIG. 1B) to diagnose attention disorder, emotional dysregulation and/or impulsive dysregulation, as further described herein.

Reference is now made to FIG. 2, which is a block diagram of a nonlimiting example of a system for diagnosing attention disorder, emotional dysregulation and/or impulsive dysregulation according to some embodiments of the invention. A system 200 may include a main microcontroller (MCU) 210 for controlling and supervising the diagnosis methods. System 200 may further include an audio stimulator 220 and a startle response detector 230.

Audio stimulator 220 may include earphones 221 for providing the audio stimuli to the examinee. Audio stimulator 220 may further include a digital gain 224 for producing audio sounds, an audio amplifier 222 for amplifying the sound to the desired intensity (e.g., having 60-120 dB) a gate 226 for controlling the duration of the sound provision, and a protection circuit 228 for limiting the sound, for example, to maximum 118 dB for 200 milliseconds if the sound is above 112 dB. Gate 226 may provide the sounds to earphones 221 at a known intensity and/or known duration.

MCU 210 may control the provision of various audio stimulations to the examinee by controlling various components of audio stimulator 220. For example, MCY 210 may control a white noise MCU 212 to provide predetermined continuous background noise. In some embodiments, startle response detector 230 may include EMG 232 and gelled EMG electrodes 235. For example, Ag—AgCl lead electrodes may be used using 4 mm/12 mm adhesive disk with signal gel.

In some embodiments, system 200 may further include a power supply 240 configured to provide, for example, an input voltage of 6-12 V. Power supply 240 may be a battery (e.g., a rechargeable battery).

In some embodiments, system 200 may further include one or more communication channels 219 for communication with external computing devices, such as, a laptop computer, personal computer, dedicated software on a mainframe computer, and the like.

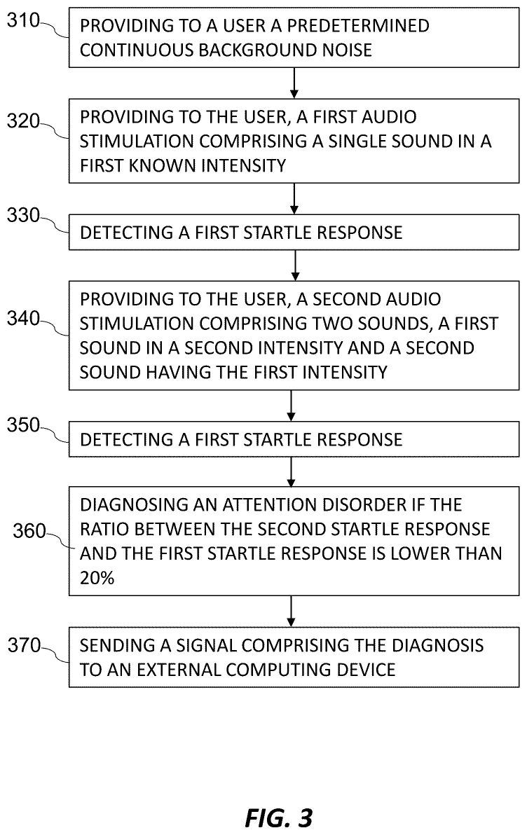

Reference is now made to FIG. 3 which is a flowchart of a method for diagnosing attention disorder according to some embodiments of the invention. The method of FIG. 3 may be conducted by computing device 10 of system 100 and/or MCU 210 of system 200 or by any other suitable controller.

In step 310, a user (e.g., an examinee) may be provided with predetermined continuous background noise. For example, stimulation units 20 or 220 may provide continuous predetermined background noise of between 40-80 dB (e.g., 40, 45, 50, 55, 60, 65, 70, 75 and 80 dB). In some embodiments, white noise MCU 212 may control digital gain 224 and amplifier 222 to provide the required background noise. In some embodiments, the continuous background noise may jam uncontrolled noise coming from the surroundings.

In step 320, the user may be provided with a first audio stimulation comprising a single sound in a first known intensity. In some embodiments, the single sound of the first audio stimulation may be provided in a known first duration. For example, stimulation units 20 or 220 may provide a single sound having first known intensity between 4-400 dB, for example, 10 dB, 43 dB, 80 dB, 96 dB, 102 dB, 108 dB, 114 dB, 140 dB, 200 dB, 300 dB, 400 dB, or any value in between. In some embodiments, the duration of the single sound may be 10-500 milliseconds.

In step 330, a first startle response may be detected. In some embodiments, startle response detectors 30 or 230 may detect the first startle response. For example, the first startle response may be detected using electromyography (EMG) electrodes placed on an Orbicularis Oculi muscle of the user, by detecting at least one of: the maximum intensity of an EMG signal, the rise in time of the EMG signal intensity, and the decay time of the EMG signal intensity and the like. In another example, the first startle response may be detected using stream of images of at least a portion the user's face, by detecting a movement (e.g., maximum movement) of an eyelid of at least one eye detected from the stream of images.

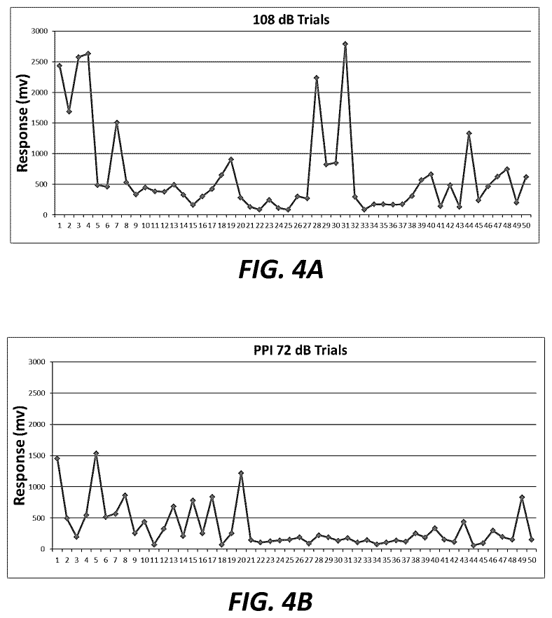

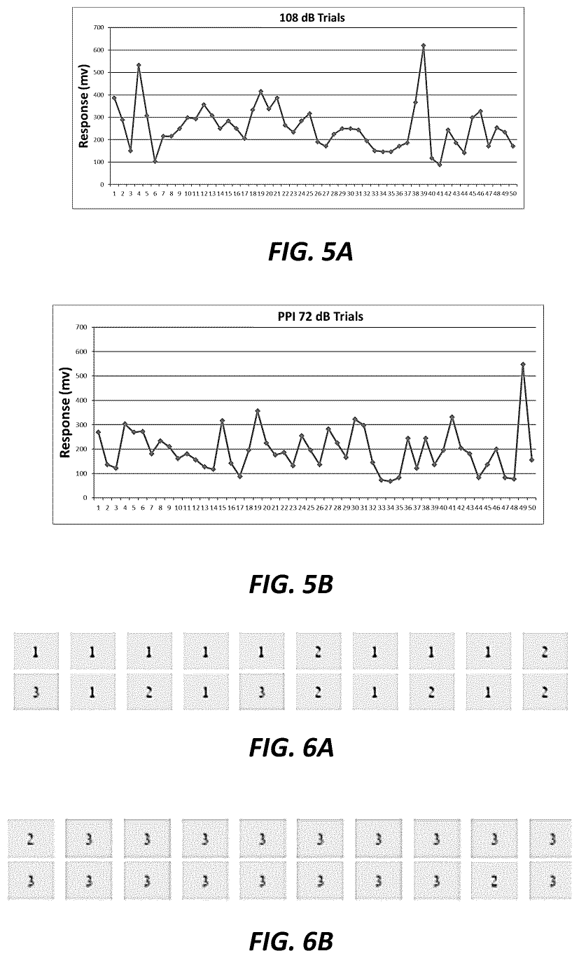

Nonlimiting examples for first startle responses detected using EMG are illustrated in the graphs in FIGS. 4A and 5A for two different users. Both users were provided with a single signal of 108 dB for 40 milliseconds.

In step 340, a second audio stimulation comprising two sounds may be provided to the user. The second audio stimulation may include a first sound in a second intensity and a second sound having the first intensity. In some embodiments, the second audio stimulation may form the PPI neurological phenomenon. In some embodiments, the second sound of the second audio stimulation may be provided in the known first duration. In some embodiments, the first sound of the second audio stimulation is provided in a second known duration.

In some embodiments, the second intensity may be selected to be higher than the background noise in an amount that allows the first sound to be noticed by the user. In some embodiments, stimulation units 20 or 220 may provide the first sound at, for example, 4-400 dB for 10-500 milliseconds, and the second sound having the first known intensity of between 4-400 dB for 2-400 milliseconds. In some embodiments, the second sound may be provided 30-600 milliseconds after the first sound. In some embodiments, the first and second stimulations may be provided to both ears of the user situationally using earphones.

In step 350, a second startle response may be detected. In some embodiments, startle response detectors 30 or 230 may detect the second startle response. For example, the second startle response may be detected using electromyography (EMG) electrodes placed on an Orbicularis Oculi muscle of the user, by detecting at least one of: the maximum intensity of an EMG signal, the rise in time of the EMG signal intensity, and the decay time of the EMG signal intensity. In another example, the second startle response may be detected using the stream of images of at least a portion of the user's face, by detecting a movement of an eyelid of at least one eye detected from the stream of images.

Nonlimiting examples for second startle responses detected using EMG are illustrated in the graphs in FIGS. 4B and 5B for two different users. Both users were provided with the first signal of 72 dB following by the second signal of 108 dB for 40 milliseconds.

In step 360, an attention disorder may be diagnosed if the reduction in the second startle response is lower in comparison to the first startle response. For a non-limiting example, referring to FIGS. 4A and 4B were the reductions of the second startle response in comparison to the first startle response is 47%, therefore, the first user was not diagnosed with an attention disorder. In another non-limiting example, given in FIGS. 5A and 5B, the reduction of the second startle response in comparison to the first startle response is 22%, which is only slightly above the lower limit for “normal” attention. In such case, the test may be repeated, as discussed with respect to the tables of FIGS. 6A and 6B.

In step 370, a signal comprising the diagnosis may be sent to an external computing device, for example, a device associated with the user, a professional, and the like, for example, via communication channel(s) 219.

In some embodiments, for a more accurate diagnosis of attention disorder a plurality of tests each repeating at least steps 310-360, may be conducted, using, for example, different first intensity levels. The results for each test (step 360) may be given a score indicating, for example, if no attention disorder (e.g., the diagnosis of FIGS. 4A and 4B) was detected the test may be given a score ‘1’. If a nonconclusive attention disorder may be detected the test may be given a score ‘2’ and if a definite attention disorder was detected the test may be given a score ‘3’. In a non-limiting example, a user may be diagnosed with an attention disorder is more than 50% of his tests were given the score ‘3’ and/or more than 80% of his tests were given the score ‘2’. Scoring tables for a “normal” user and a user diagnosed with attention disorder are given in FIGS. 6A and 6B respectively.

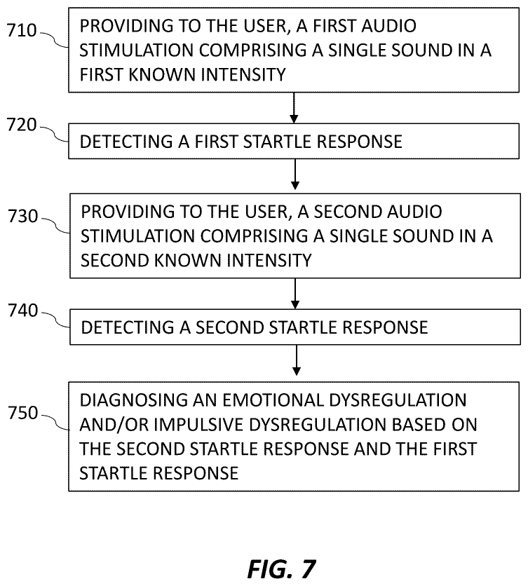

Reference is now made to FIG. 7 which is a flowchart of a method for diagnosing emotional dysregulation and/or impulsive dysregulation according to some embodiments of the invention. The method of FIG. 7 may be conducted by computing device 10 of system 100 and/or MCU 210 of system 200 or by any other suitable controller.

In step 710, the user may be provided with a first audio stimulation comprising a single sound in a first known intensity. In some embodiments, the single sound of the first audio stimulation may be provided in a known first duration. For example, stimulation units 20 or 220 may provide a single sound having first known intensity between 4-400 dB, for example, 10 dB, 43 dB, 80 dB, 96 dB, 102 dB, 108 dB, 114 dB, 140 dB, 200 dB, 300 dB, 400 dB, or any value in between. In some embodiments, the duration of the single sound may be 10-500 milliseconds.

In step 720, a first startle response may be detected. In some embodiments, startle response detectors 30 or 230 may detect the first startle response. For example, the first startle response may be detected using electromyography (EMG) electrodes placed on an Orbicularis Oculi muscle of the user, by detecting at least one of: the maximum intensity of an EMG signal, the rise in time of the EMG signal intensity, and the decay time of the EMG signal intensity. In another example, the first startle response may be detected using a stream of images of at least a portion of the user's face, by detecting a movement of an eyelid of at least one eye detected from the stream of images.

In step 730, the user may be provided with a second audio stimulation comprising a second sound in a second known intensity higher than the first intensity. In some embodiments, the first sound of the first audio stimulation and the second sound of the second audio stimulation are provided in a known first duration, for example, 10-500 milliseconds. In some embodiments, the first known intensity is between 4-400 dB and the second known intensity is between 4-400 dB. For example, if the first sound has an intensity of 102 dB, the second sound may have an intensity of 108 dB. In some embodiments, the duration between the first and second stimulation (e.g., the inter-stimulus interval) is 30-600 milliseconds. In some embodiments, the first and second stimulations may be provided to both ears of the user situationally using earphones.

In step 740, a second startle response may be detected. In some embodiments, startle response detectors 30 or 230 may detect the second startle response. For example, the second startle response may be detected using electromyography (EMG) electrodes placed on an Orbicularis Oculi muscle of the user, by detecting at least one of: the maximum intensity of an EMG signal, the rise in time of the EMG signal intensity, and the decay time of the EMG signal intensity. In another example, the second startle response may be detected using the stream of images of at least a portion the user's face, by detecting a maximum movement of an eyelid of at least one eye detected from the stream of images.

In step 750, emotional dysregulation and/or impulsive dysregulation may be diagnosed based on the first and second startle responses, for example, if the second startle response is equal to or lower than the first startle response. In some embodiments, for all users not suffering from any emotional dysregulation and/or impulsive dysregulation the second startle response should be higher than the first startle response since a higher stimulation was provided. In some embodiments, the higher the stimulation intensity the higher should be the second startle response.

In some embodiments, the user may be provided with a third audio stimulation comprising a third sound in a third known intensity higher than the second intensity. For example, if the first sound has an intensity of 102 dB, the second sound may have an intensity of 108 dB and the third sound may have an intensity of 114 dB. In some embodiments, a third startle response may be detected, and diagnosing the emotional dysregulation and/or impulsive dysregulation may include determining if the third startle response is equal to or lower than the second startle response.

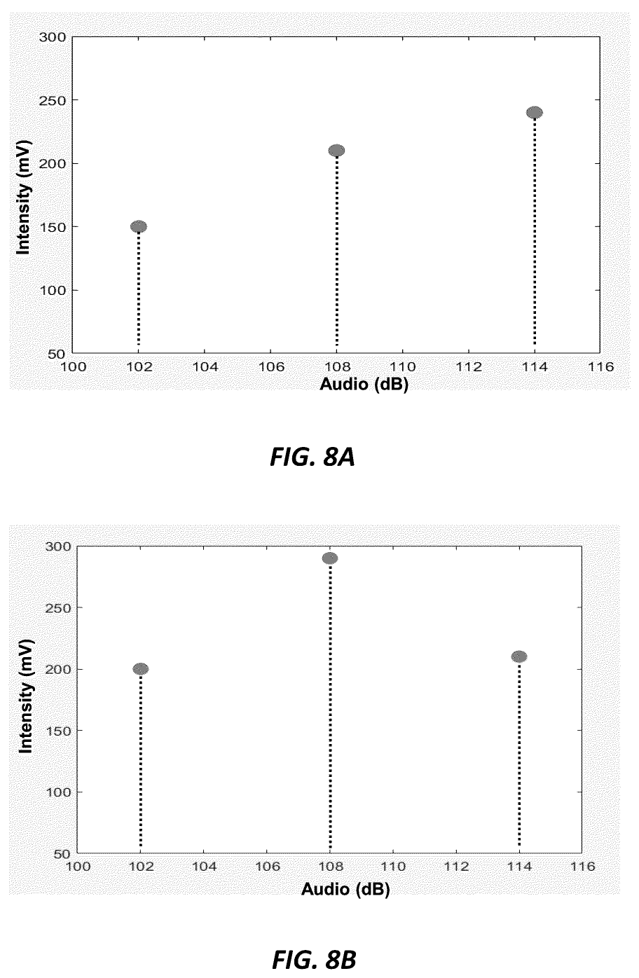

Reference is now made to FIGS. 8A and 8B which include graphs presenting the intensity of the startle response measured for two examinees in response to the provision of three audio stimulation having increasing intensities. In FIG. 8A the maximum intensity of the startle response for a normal examinee is presented. The Examiner was exposed to three audio stimulations having 102, 108, and 114 dB. As clearly shown in the graph, the higher the audio stimulation intensity, the higher the startle response. The startle response of a second examine, exposed to the same audio stimulations is presented in FIG. 8B. As clearly shown in the graph, the third startle response (200 mV) is much lower than the second startle response (300 mV) although the examinee was provided with a higher audio stimulation, The second examinee was diagnosed with emotional dysregulation and/or impulsive dysregulation.

Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method for diagnosing attention disorder, comprising:
providing to a user a predetermined continuous background noise;
providing to the user, a first audio stimulation comprising a single sound in a first known intensity;
detecting a first startle response;
providing to the user, a second audio stimulation comprising two sounds, a first sound in a second intensity and a second sound having the first intensity, wherein the second intensity is selected to be higher than the background noise in an amount that allows the first sound to be noticed by the user;
detecting a second startle response;
diagnosing an attention disorder by measuring the inhibition value between the second startle response and the first startle response compared to the inhibition value of the healthy population.
sending a signal comprising the diagnosis to an external computing device.

2. The method of claim 1, wherein the single sound of the first audio stimulation and the second sound of the second audio stimulation are provided in a known first duration.

3. The method of claim 1, wherein the first sound of the second audio stimulation is provided in a second known duration.

4. The method according to claim 1, wherein the first startle response and the second startle response are detected using electromyography (EMG) electrodes placed on an Orbicularis Oculi muscle of the user.

5. The method of claim 4, wherein detecting each of the first startle response and detecting the second startle response comprises detecting at least one of: the maximum intensity of an EMG signal, the rise in time of the EMG signal intensity and the decay time of the EMG signal intensity.

6. The method according to claim 1, wherein the first startle response and the second startle response are detected using stream of images of at least a portion the user's face.

7. The method of claim 6, wherein detecting each of the first startle response and detecting the second startle response comprises detecting a maximum movement of an eyelid of at least one eye detected from the stream of images.

8. The method according to claim 1, wherein the first known intensity is between 4-400 dB.

9. The method according to claim 1, wherein the predetermined continuous background noise is between 40-80 dB.

10. The method according to claim 1, wherein the second sound is provided 30-600 milliseconds after the first sound.

11. The method according to claim 1, wherein the first and second audio stimulations are provided to both ears of the user situationally using earphones.

12. The method according to claim 1, wherein the duration of the first audio stimulation and the second sound in the second audio stimulation is 10-500 milliseconds.

13. The method claim 1, wherein the duration of the second sound is 2-400 milliseconds.

14. A method for diagnosing emotional dysregulation and/or impulsive dysregulation comprising:
providing to a user, a first audio stimulation comprising a first sound in a first known intensity;
detecting a first startle response;
providing to the user, a second audio stimulation comprising a second sound in a second known intensity higher than the first intensity;
detecting a second startle response;
diagnosing an emotional dysregulation and/or impulsive dysregulation if the second startle response is equal to or lower than the first startle response; and
sending a signal comprising the diagnosis to an external computing device.

15. The method of claim 14, further comprising providing to a user a predetermined continuous background noise.

16. The method of claim 14, wherein the first sound of the first audio stimulation and the second sound of the second audio stimulation are provided in a known first duration.

17. The method according to claim 14, wherein the first sound of the second audio stimulation is provided in a second known duration.

18. The method according to claim 14, wherein the first startle response and the second startle response are detected using electromyography (EMG) electrodes placed on an Orbicularis Oculi muscle of the user.

19. The method of claim 18, wherein detecting each of the first startle response and detecting the second startle response comprises detecting at least one of: the maximum intensity of an EMG signal, the rise in time of the EMG signal intensity and the decay time of the EMG signal intensity.

20. The method according to claim 14, wherein the first startle response and the second startle response are detected using stream of images of at least a portion the user's face.

21.-26. (canceled)
